(19) United States
(12) Patent Application Publication
(10) Pub. No.: US 2022/0184324 A1
(43) Pub. Date: Jun. 16, 2022

(54) A FLUID PRODUCT INJECTION DEVICEz

(71) Applicant: APTAR FRANCE SAS, LE NEUBOURG (FR)

(72) Inventor: Ludovic PETIT, VITOT (FR)

(73) Assignee: APTAR FRANCE SAS, LE NEUBOURG (FR)

(21) Appl. No.: 17/442,889

(22) PCT Filed: Apr. 3, 2020
(86) PCT No.: PCT/FR2020/000088

§ 371 Date: Sep. 24, 2021

(30) Foreign Application Priority Data
Apr. 4, 2019 (FR) 1903626

(51) Int. Cl. A61M 5/50; A61M 5/315; A61M 5/32

(52) U.S. Cl. CPC A61M 5/508; A61M 5/3269; A61M 5/31511

(57) ABSTRACT

Disclosed is a fluid product injection device comprising: —a syringe comprising a vessel (1) containing fluid product to be injected, a piston (3) being arranged so as to be axially movable in the vessel (1) and a needle (2) provided with an injection tip (20) being attached to the vessel (1), —a piston rod (5) axially movable relative to the vessel (1) and cooperating with the piston (3) during injection in order to move it axially in the vessel (1), —a sleeve (10) arranged around the syringe, being axially movable relative to the vessel (1) between an idle position, in which the sleeve (10) does not cover the injection tip (20) of the needle (2), and a projecting position, in which the sleeve (10) covers the injection tip (20) of the needle (2), the sleeve (10) being in its idle position before actuation of the injection device, the sleeve (10) being moved towards its projecting position after the injection of the fluid product, the sleeve (10) being moved by the piston rod (5).

The present invention relates to a fluid injection device, and more particularly to such a device provided with a safety device.

Safety devices for injection syringes, in particular syringes of the pre-filled type, are well known. Document FR 2 922 112 describes an example of such a prior-art device.

Such safety devices generally comprise an outer sleeve that is assembled around the syringe, and that is adapted to be deployed at the end of injection so as to cover the needle. Such devices generally require numerous movable and/or deformable parts, such as springs and flexible tabs, thereby making manufacture and assembly relatively complex. The use of one or more springs and deformable plastic parts may generate risks of malfunction, in particular after a long storage time, during which the spring may lose its dynamic force and the flexible tabs may lose their flexibility or elasticity.

Documents US 2004/171 991, U.S. Pat. Nos. 6,428,519, 6,110,147 and 5,151,088 describe other prior-art devices. All these devices provide a movable sleeve disposed inside the syringe and adapted to be pushed manually at the end of the actuating stroke towards a safety position in which it covers the needle. Such devices are complex and thus costly to manufacture and to assemble.

An object of the present invention is to provide an injection device that does not have the above-mentioned drawbacks.

Another object of the present invention is to provide an injection device that is reliable in use, that is safe and that prevents any risk of injury, and that is simple and inexpensive to manufacture, to assemble, and to use.

The present invention thus provides a fluid product injection device comprising:
a syringe comprising a reservoir containing fluid product to be injected, a piston being arranged axially movable in said reservoir and a needle provided with an injection tip being fixed to said reservoir,
a piston rod that can be moved axially relative to said reservoir and that co-operates with said piston during injection so as to move it axially in said reservoir,
a sleeve that is arranged around said syringe so as to be movable axially relative to said reservoir between a rest position in which said sleeve does not cover said injection tip of said needle, and a projected position in which said sleeve covers said injection tip of said needle, said sleeve being in its rest position before actuation of the injection device,

said sleeve being moved towards its projected position after said fluid product has been injected, said sleeve being moved by said piston rod.

Advantageously, in the rest position, said sleeve is connected to said syringe, in particular by breakable bridges.

Advantageously, a second piston is arranged to move axially in the reservoir, said fluid product to be injected being arranged between said piston and said second piston.

Advantageously, said second piston is perforated by said needle at the start of actuation.

Advantageously, said piston is perforated by said needle at the end of injection.

Advantageously, said sleeve comprises internal tabs that extend inside the syringe and tabs or an outer sleeve that extends outside the syringe.

Advantageously, said device does not have a spring.

These characteristics and advantages and others of the present invention appear more clearly from the following detailed description, given by way of non-limiting example, and with reference to the accompanying drawings, and in which:

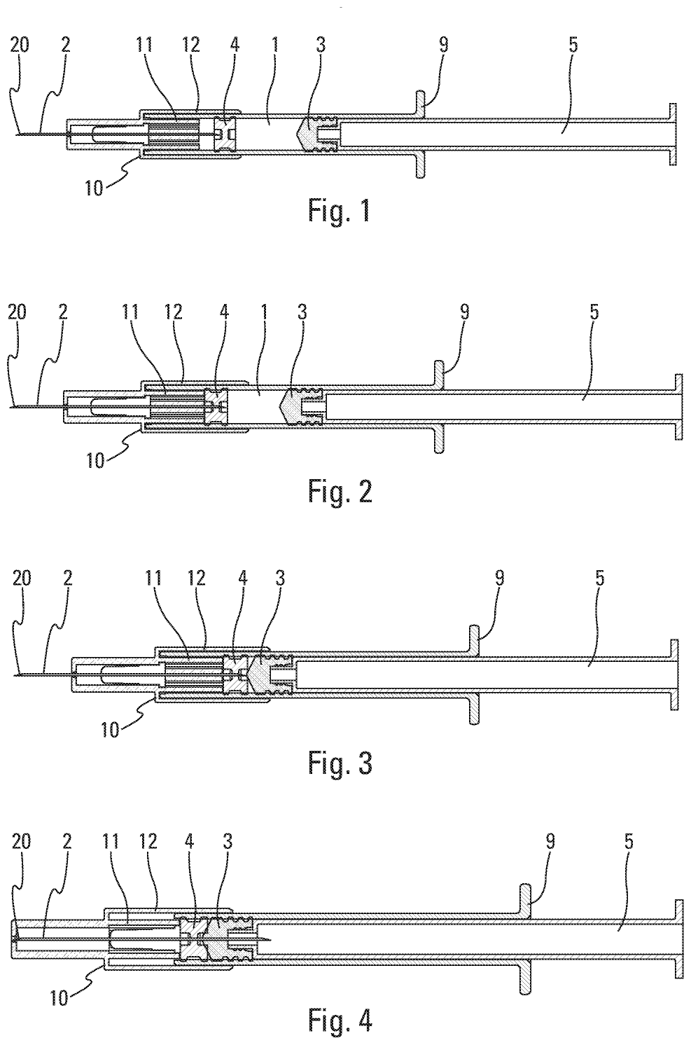

FIG. 1 is a diagrammatic section view of an injection device in an advantageous embodiment, in its position before injection,

FIG. 2 is a view similar to the view in FIG. 1, during injection,

FIG. 3 is a view similar to the view in FIG. 2, at the end of injection, before the safety device has been triggered;

FIG. 4 is a view similar to the view in FIG. 3, after the safety device has been triggered.

The manual injection device shown in the figures includes a syringe comprising a reservoir 1 containing the fluid to be injected, a needle 2 that is fastened to said reservoir 1 and through which the fluid is dispensed, and a piston 3 that is adapted to be moved in said reservoir 1 so as to perform the injection.

The needle 2 includes an injection tip 20 that is jabbed into the injection zone during use.

A piston rod 5 co-operates with said piston 3 during injection so as to move it in the reservoir 1.

Preferably at its distal end, the syringe may be provided in conventional manner with a radial collar 9 against which the user can bear when actuating the piston rod 5.

Advantageously, a second piston 4 is provided in the reservoir 1, the fluid product to be dispensed being arranged between the piston 3 and the second piston 4. The function of the second piston 4 is to isolate the fluid product before use, and it is perforated by the needle 2 at the start of actuation. The second piston 4 is also axially movable in said reservoir 1 during actuation.

A sleeve 10 is provided around the syringe, being axially movable relative to the syringe between a rest position and a projecting position. This sleeve 10 comprises internal tabs 11 that extend inside the syringe and tabs or an outer sleeve 12 that extends outside the syringe. This embodiment, which minimizes the quantity of material of the sleeve 10 that must be housed inside the syringe, makes it possible to simplify manufacture and assembly. Furthermore, this makes it possible to minimize the dimensions, in particular radial dimensions, of the syringe. Thus, while the devices in which the entire sleeve is received inside the syringe require sufficient dimensions to do this, the present invention makes it possible to reduce these dimensions, since only the internal tabs 11 of the sleeve 10 are arranged inside the syringe.

Before injection, the sleeve 10 is arranged around the reservoir 1 in its rest position, in such a manner as to expose the injection tip 20 of the needle 2. It should be observed that before use, the injection tip 20 may be protected by an appropriate cap (not shown) that the user removes when it is desired to use the injection device.

After injection, said sleeve 10 is moved axially relative to the reservoir 1 towards its projecting position in which it is arranged around the injection tip 20 of the needle 2, so as to avoid any risk of injury with said needle 2, thereby forming a post-injection safety device.

With reference to FIGS. 1 to 4, the user who wishes to use the injection device jabs the injection site with the injection tip 20 of the needle 2, and then exerts axial pressure on the piston rod 5. Said piston rod moves axially relative to the reservoir 1, so as to move the piston 3 in the reservoir 1. This generates pressure in the fluid product, and since the fluid product is incompressible, it transmits the pressure to the second piston 4. The second piston is thus forced to move axially against the needle 2, which thus comes to perforate it, as can be seen in FIG. 2. From that moment on, axial movement of the piston rod 5 moves the piston 3 in the reservoir 1, thereby expelling the fluid product through the needle 2 so as to inject it into the injection site.

At the end of injection, the piston 3 arrives in abutment against the second piston 4, itself in abutment against the internal tabs 11 of the sleeve 10, as can be seen in FIG. 3. If the user continues to press axially on the piston rod 5, this pressing force will be transmitted via the piston 3 and the second piston 4 to said sleeve 10. The sleeve 10 is then moved towards its projecting position in which it covers the injection tip 20 of the needle 2, so as to avoid any risk of injury with the needle 2, as can be seen in FIG. 4. Simultaneously, the needle 2, that does not move relative to the reservoir 1, perforates the piston 3 to allow the axial movement of the sleeve 10.

Advantageously, in the rest position, the sleeve 10 may be connected to the syringe, e.g. by breakable bridges made by molding. The breakable bridges are broken at the end of injection, when the sleeve 10 is moved towards its projecting position. Thus, during the axial movement of the piston rod 5 relative to the reservoir 1, it is guaranteed that initially the fluid product is injected through the needle 2, and that the sleeve 10 is moved only once injection has finished. The sleeve 10 may be connected to the syringe in the rest position in other ways, e.g. by friction or by mechanical jamming.

An advantage of the present invention is the absence of spring and flexible tab(s), thereby simplifying the manufacture and assembly of the device, and makes its use more reliable.

Although the present invention is described above with reference to an advantageous embodiment, naturally various modifications can be applied thereto by the person skilled in the art, without going beyond the ambit of the present invention, as defined by the accompanying claims.

1. A fluid product injection device comprising:
a syringe comprising a reservoir (1) containing fluid product to be injected, a piston (3) being arranged so as to be axially movable in said reservoir (1) and a needle (2) provided with an injection tip (20) being attached to said reservoir (1),
a piston rod (5) axially movable relative to said reservoir (1) and cooperating with said piston (3) during injection in order to move it axially in said reservoir (1),
a sleeve (10) arranged around said syringe while being axially movable relative to said reservoir (1) between a rest position, in which said sleeve (10) does not cover said injection tip (20) of said needle (2), and a projecting position, in which said sleeve (10) covers said injection tip (20) of said needle (2), said sleeve (10) being in its rest position before actuation of the injection device,
characterized in that said sleeve (10) is moved towards its projecting position after injection of said fluid product, said movement of said sleeve (10) being performed by said piston rod (5).

2. The device according to claim 1, wherein, in its rest position, said sleeve (10) is connected to said syringe, in particular by breakable bridges.

3. The device according to claim 1, wherein a second piston (4) is arranged to move axially in the reservoir (1), said fluid product to be injected being arranged between said piston (3) and said second piston (4).

4. The device according to claim 3, wherein said second piston (4) is perforated by said needle (2) at the start of actuation.

5. The device according to claim 1, wherein said piston (3) is perforated by said needle (2) at the end of injection.

6. The device according to claim 1, wherein said sleeve (10) includes internal tabs (11) that extend inside the syringe and tabs or an outer sleeve (12) that extends outside the syringe.

7. The device according to claim 1, wherein said device does not have a spring.
